(19) United States
(12) Patent Application Publication
(10) Pub. No.: US 2014/0295573 A1
(43) Pub. Date: Oct. 2, 2014

(54) BIOSENSOR WITH DUAL GATE STRUCTURE AND METHOD FOR DETECTING CONCENTRATION OF TARGET PROTEIN IN A PROTEIN SOLUTION

(71) Applicant: NATIONAL TAIWAN UNIVERSITY, Taipei City (TW)

(72) Inventors: Jian-jang HUANG, Taipei (TW); Tsung-Lin YANG, Taipei City (TW); Yi-Chun SHEN, Taipei (TW); Chun-Hsu YANG, Taipei (TW)

(73) Assignee: National Taiwan University, Taipei City (TW)

(21) Appl. No.: 13/850,618

(22) Filed: Mar. 26, 2013

(51) Int. Cl. G01N 33/543

(52) U.S. Cl. CPC G01N 33/5438
USPC 436/501; 422/69

(57) ABSTRACT

A biosensor with a dual gate structure is disclosed herein. The biosensor comprises: a transistor, a sensing pad, and a plurality of nanostructures. The sensing pad has a conductive area working as another gate and neighboring to the channel layer of the transistor, and a sensing area extended outward from the conductive area to be far away from the channel layer of the transistor, wherein the gate and the conductive area of the sensing pad are separated from each other by the channel layer. The plurality of nanostructures are utilized to bind a first protein to generate a drain current value, when the first protein is combined with the target protein and another drain current value is generated, whereby a variation between the two drain current values is calculated to obtain the concentration of the target protein in the protein solution.

### CROSS-REFERENCE

This invention is partly disclosed in a thesis entitled “IGZO-TFT Protein Sensors with ZnO nanorods for Enhanced Sensitivity and Specificity” on Jul. 19, 2012 completed by Yi-Chun Shen and a thesis entitled “IGZO-TFT Protein Sensors for Enhanced Sensitivity and Specificity.” on Dec. 7, 2012 completed by Chun-hsu Yang, Yi-Chun Shen, Tsung-Lin Yang, and Jian-Jang Huang.

### TECHNICAL FIELD OF THE INVENTION

The present invention relates to biosensor technology, and more particularly to, a biosensor which is applied for electrically detecting a concentration of a target protein in a protein solution.

### BACKGROUND OF THE INVENTION

By the nanoscale science and engineering, a nanoscale biosensors can be fabricated with a great performance of faster response, higher sensitivity and specificity than the past planar sensor configurations. With the nano-dimension of the biosensor, a contact surface can be dramatically expanded wider to enhance a binding effect with biological and chemical reagents for biological and biochemical applications or researches, e.g. significantly monitoring and protecting the environment.

Please refer to FIG. 1 which illustrates a traditional biosensor 100 for detecting a concentration of a target protein in a protein solution. The traditional biosensor 100 is a transistor-based biosensor 100. The transistor 110 comprises a source S and a drain D, and a sensor plate 111, which is utilized for detecting the protein solution, is disposed on the transistor 110 where the gate G position is, in order to form a sensing gate 112. The sensing gate 112 comprises a nanotip array 113, which is utilized for binding with the protein of the protein solution. Because of the structure of the transistor 110, the sensing gate 112 is located on a channel layer 114, and the channel layer 114 is between the source S and the drain D.

The existing method for detecting a concentration of a target protein in a protein solution is by measuring a variation of a drain current, the variation of the drain current is caused by a variation of charge distribution of the channel layer 114 when the target protein (e.g. antigens) combines with the protein (e.g. antibodies), which corresponds to the target protein, and the concentration of a target protein in a protein solution is measured by calculating the variation of drain current. When the biosensor 100 is applied for detecting the concentration of a target protein in a protein solution, the gate G and the drain D are applied voltage in advance, therefore the gate G and the drain D are relatively positive/negative electric potential to the source S. When the gate G-source S voltage (V_{GS}) is higher than the threshold voltage (Vₜₕ), a channel layer 114 is established, and a drain current is generated in order for the drain current to have a first current value at this time.

Antibodies, which correspond to antigens under test, are applied to the sensing gate 112 for a determined time, and then the sensing gate 112 is washed by a buffer solution, and only the antibodies which are attached on the nanotip array 113 are remained. A protein solution which includes the antigens under test is applied to the sensing gate 112 having the antibodies attached thereon, therefore the antigens under test are combined with the antibodies in order for the charge distribution of the channel layer 114 to be changed, and the drain current has a second current value at this time. By comparing the first current value of the drain current to the second current value of the drain current and calculating the difference value between them, the concentration of a target protein in a protein solution is obtained.

However, the sensing plate 111 is disposed on the transistor 110 where the gate G position is, so the sensing area (not shown) is limited by the size of the transistor 110 and the measurement of the drain current is difficult. On the other hand, the distance between the sensing gate 112 and the channel layer 114 is overly close, therefore the charge distribution of the channel layer 114 is influenced by an electromagnetic interference and hence data distortions can appear. When the magnitudes of the first current value is similar to the second current value of the drain current, the data distortions will also appear, which degrades the sensitivity of the biosensor 100. Therefore, a great amount of the protein solution under test may be required.

### SUMMARY OF THE INVENTION

An objective of the present invention is to provide a biosensor with a dual gate structure capable to raise the sensitivity of the biosensor, increase the sensing area, and prevent the gate from a charge distribution resulted from an influence of an electromagnetic interference.

To solve the above-mentioned problem, the present invention provides a biosensor with a dual gate structure for detecting a concentration of a target protein in a protein solution. The biosensor comprises a transistor comprising a gate, a source and a drain, wherein a channel layer is formed to establish electrical connection between the source and the drain; a sensing pad having a conductive area working as another gate and neighboring to the channel layer of the transistor, and a sensing area extended outward from the conductive area to be away from the channel layer of the transistor, wherein the gate and the conductive area of the sensing pad are separated from each other by the channel layer. The sensing area is utilized to apply the first protein to generate a drain current value via the transistor, when the protein solution is applied on the sensing area to combine the first protein with the target protein and another drain current value is generated via the transistor, the concentration of the target protein in the protein solution is obtained by a variation between the two drain current values.

To solve the above-mentioned problem, the present invention provides a method of detecting a concentration of a target protein in a protein solution. The method comprises steps of: forming a transistor having a gate, a source and a drain, wherein a channel layer is formed to establish an electrical connection between the source and the drain; forming a sensing pad having a conductive area working as another gate and neighboring to the channel layer of the transistor, and a sensing area extended outward from the conductive pad to be away from the channel layer of the transistor, and the gate and the conductive area of the sensing pad are separated from each other; attaching nanostructures on the sensing area; applying a specific voltage on the gate and the drain of the transistor, and the gate and the drain being relatively positive/negative electric potential to the source of the transistor; applying first proteins on the sensing area, and measuring a first current value of the drain current; applying the protein solution having the target protein on the sensing area, and measuring a second current value of the drain current; and by a variation between the first current value and the second current value, obtaining the concentration of the target protein in the protein solution.

To solve the above-mentioned problem, the present invention provides a biosensor with a dual gate structure for detecting a concentration of a target protein in a protein solution. The biosensor comprises a transistor comprising a gate, a source and a drain, wherein a channel layer is formed to establish electrical connection between the source and the drain; a sensing pad having a conductive area working as another gate and neighboring to the channel layer of the transistor, and a sensing area extended outward from the conductive area to be away from the channel layer of the transistor, wherein the gate and the conductive area of the sensing pad are separated from each other. The sensing area is utilized to apply a first protein to generate a drain current value via the transistor, when the protein solution is applied on the sensing area to combine the first protein with the target protein and another drain current value is generated via the transistor, the concentration of the target protein in the protein solution is obtained by a variation between the two drain current values.

Contrary to the existing technique, because the sensing pad is extended outward form the transistor, the size of the sensing pad is designed according to requirements of a user. The transistor thus has a dual gate structure, so that the control of the gate voltage is more sensitive. When the magnitude of the measured first current value and the measured second current value of the drain current is very close, the user can adjust the gate voltage so that two current values can be distinguished, which ensures a great sensitivity of the biosensor in the present invention.

For better understanding of the aforementioned content of the present invention, the preferred embodiments are described in detail in conjunction with the appending figure as follows.

### BRIEF DESCRIPTION OF THE DRAWINGS

FIG. 1 illustrates a diagram of a traditional biosensor for detecting a concentration of a target protein in a protein solution;

FIG. 2A illustrates a structural diagram of a biosensor according to a first embodiment of the present invention;

FIG. 2B illustrates a cross-sectional diagram of the biosensor according to an A-A′ split line shown in FIG. 2A;

FIG. 2C illustrates another diagram of the biosensor after removing a photoresist shown in FIG. 2A;

FIG. 2D illustrates a cross-sectional diagram of the biosensor according to another embodiment of the present invention.

FIG. 3 illustrates the flow chart of a method of detecting a concentration of a target protein in a protein solution;

FIG. 4A illustrates a drawing of drain currents versus gate voltages for a prior biosensor as structured in a bare biosensor;

FIG. 4B illustrates a drawing of drain currents versus gate voltages for the biosensor shown in FIG. 2C as structured in a functionalized biosensor;

FIG. 4C illustrates a drawing of drain currents versus drain voltages for the bare biosensor described in FIG. 4A; and

FIG. 4D illustrates a drawing of drain currents versus drain voltages for the functionalized biosensor described in FIG. 4B.

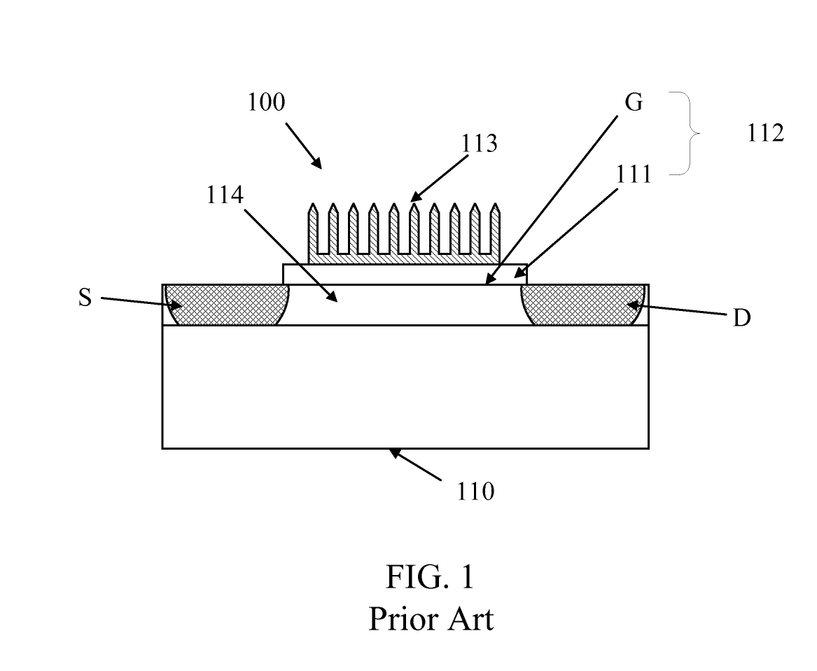

### DETAILED DESCRIPTION OF THE INVENTION

Hereinafter, exemplary embodiments of the present invention will be described with reference to the accompanying drawings. In the following description, the same elements will be designated by the same reference numerals although they are shown in different drawings. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if it is described in the specification that one component is “connected,” “coupled” or “joined” to another component, a third component may be “connected,” “coupled,” and “joined” between the first and second components, although the first component may be directly connected, coupled or joined to the second component.

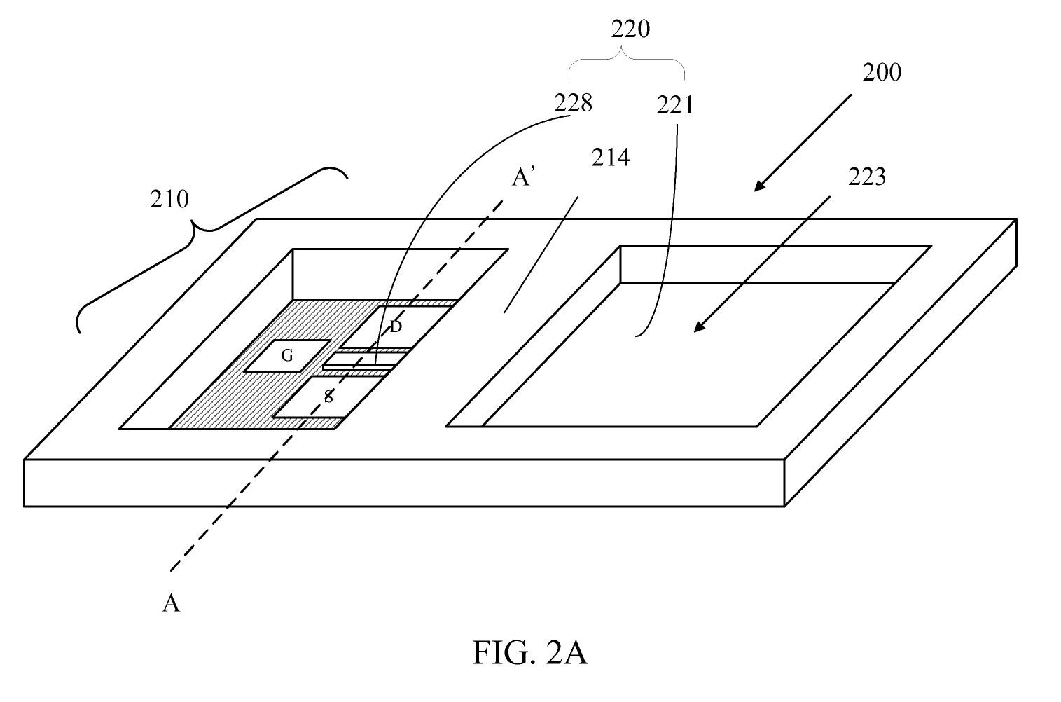

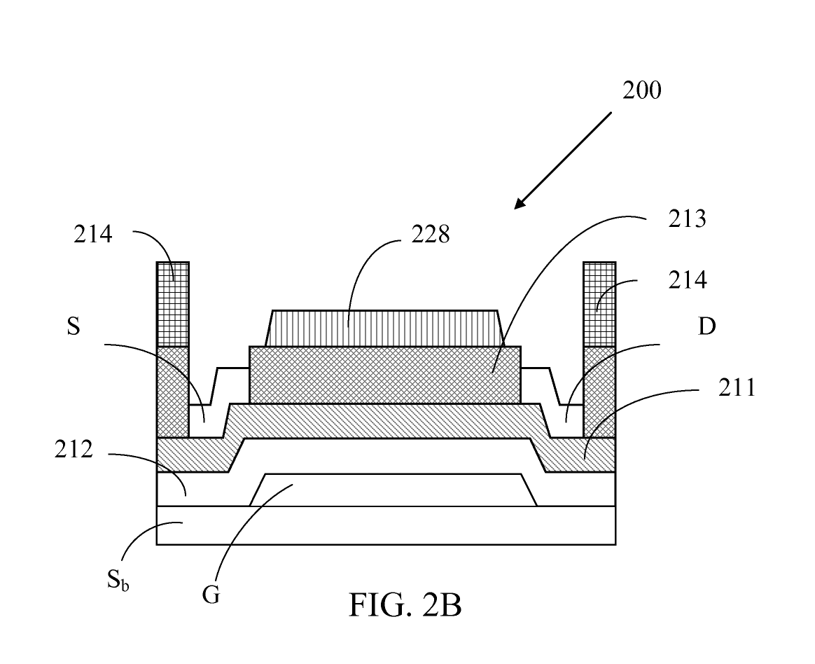

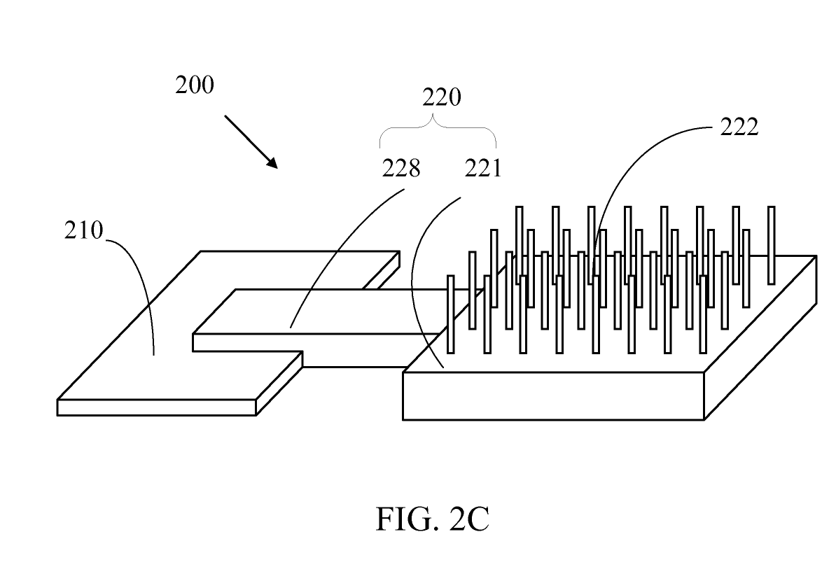

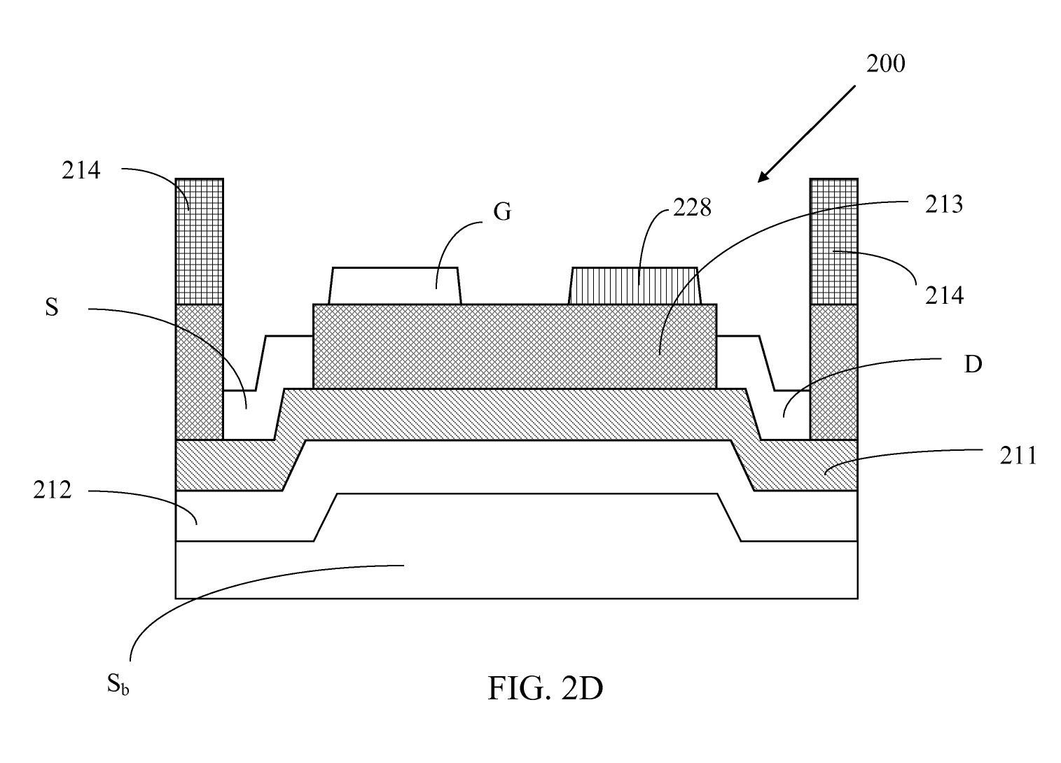

Firstly, FIG. 2A illustrates a structural diagram of a biosensor 200 according to a first embodiment of the present invention, FIG. 2B illustrates a cross-sectional diagram of the biosensor 200 according to an A-A′ split line shown in FIG. 2A, and FIG. 2C illustrates another diagram of the biosensor 200 after a photoresist 214 shown in FIG. 2A is removed. The biosensor 200 with a dual gate structure of the present invention is utilized for detecting a concentration of a target protein in a protein solution. As shown in FIGS. 2A and 2B, the biosensor 200 primarily includes a transistor 210 and a sensing pad 220. The transistor 210 has a substrate S_{b}, a source S, a drain D and a gate G, wherein an insulating layer 212 is formed above the gate G, the gate G is formed above the substrate S_{b}, the substrate S_{b} is located on a bottom of the transistor 210, and a channel layer 211 is formed above the insulating layer 212 to establish electrical connection between the source S and the drain D. In some embodiments, the transistor 210 may be realized as a TFT (thin film transistor), a MOSFET (metal oxide semiconductor field effect transistor), or an HEMT (high electron mobility transistor) and the insulating layer 212 may be made from a titanium dioxide (TiO₂). The sensing pad 220 is disposed above the channel layer 211 and is divided into a conductive area 228 working as another gate and neighboring to the channel layer 211 of the transistor 210, and a sensing area 221 (see FIG. 2A) integrally extended outward from the conductive area 228 to be away or isolated from the channel layer 211 of the transistor 210. Therefore, the gate G and the conductive area 228 of the sensing pad 220 are separated from each other by the channel layer 211 vertically (gate G and conductive area 228 are separated vertically with the channel layer 211 sandwiched in between(please see FIG. 2B)) or horizontally (gate G and conductive are 228 are located on the same plane of the channel layer 211 (please refer FIG. 2D)) so that dual gate structure in the transistor 210 is formed. The sensing area 221 can be sized on various user demands for carrying and electrically detecting a protein solution thereon, wherein the sensing pad 220 is made from the good conductive metals, including but not limited to, for example, gold (Au), silver (Ag) and copper (Cu), by alone or combination thereof. A passivation layer 213 is formed between the conductive area 228 of the sensing pad 220 and the channel layer 211.

Further referring to FIG. 2C, a plurality of nanostructures 222 according to the first embodiment of the present invention, are applied to the sensing area 221 by electrostatical attachment. In this embodiment, the nanostructures 222 may be made from ZnO nanorods, TiO₂ nanorods or other types of oxide materials which do not harm the proteins, by combination or alone. The nanostructures 222 are utilized for improving a binding ability between the sensing area 221 of the sensing pad 220 and the proteins.

Further referring to FIG. 2A, the transistor 210 and the sensing area 221 of the sensing pad 220 are surrounded by electrically isolating materials (such as photoresist or polymer) 214 to form a sensing sink 223 which is utilized for containing the protein solution and isolating the protein solution from the transistor 210 so as to avoid the damage to the transistor 210 that is resulted from dipping in the solution. By the structure of the sensing sink 223, the sensing area 221 is increased significantly and thus increases the sensitivity of detection. Preferably, the volume of the sensing sink 223 is 72.75 nl (nanoliter).

A method of measuring a variation of a drain current is applied on the biosensor 200 to calculate the concentration of the target protein in the protein solution, the variation of the drain current is resulted from a variation of charge distribution of the channel layer 211 when the protein of the protein solution (i.e. antigens) combines with the protein carried on the sensing area 221 of the sensing pad 220 (i.e. antibodies), so that the concentration of the target protein in the protein solution can be obtained by calculating the variation of said drain current.

Further referring to FIG. 2D, FIG. 2D illustrates a cross-sectional diagram of the biosensor according to another embodiment of the present invention. The biosensor 200 in this embodiment of the present invention is similar with the biosensor 200 in FIG. 2B. Therefore, the same indicator and name are followed. The difference between FIG. 2D and FIG. 2B is the gate G is disposed on one side of the channel 211 where the conductive area 228 is disposed. The operation processes in FIG. 2D are the same as FIG. 2B, so that the operation processes are not be repeated herein.

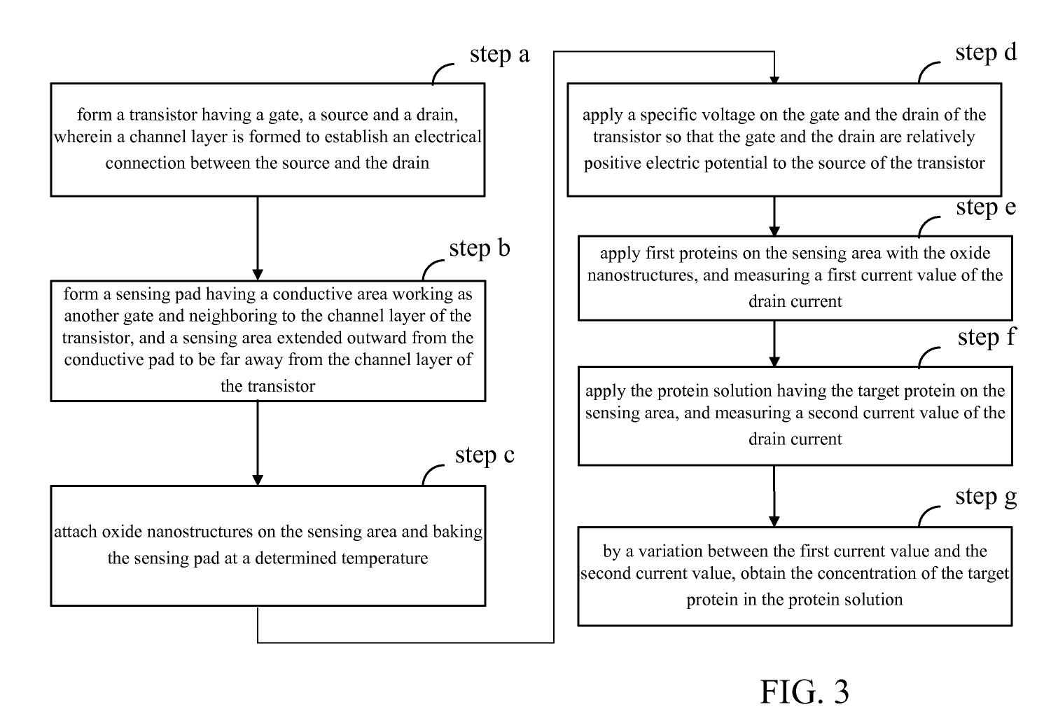

Further referring to FIG. 3, a flow chart of a method of detecting a concentration of a target protein in a protein solution is illustrated herein. The method comprising: step a) forming a transistor having a gate, a source and a drain, wherein a channel layer is formed to establish an electrical connection between the source and the drain; step b) forming a sensing pad having a conductive area working as another gate and neighboring to the channel layer of the transistor, and a sensing area extended outward from the conductive pad to be far away from the channel layer of the transistor, wherein the gate and the conductive area of the sensing pad are separated from each other by the channel layer; step c) attaching nanostructures on the sensing area; step d) applying a specific voltage on the gate and the drain of the transistor so that the gate and the drain are relatively positive/negative electric potential to the source of the transistor; step e) applying first proteins on the sensing area with the nanostructures, and measuring a first current value of the drain current; step f) applying the protein solution having the target protein on the sensing area, and measuring a second current value of the drain current; and step g) by a variation between the first current value and the second current value, obtaining the concentration of the target protein in the protein solution.

In this embodiment, the main experimental subjects are EGFR (epidermal growth factor receptor) antibodies and EGFR antigens.

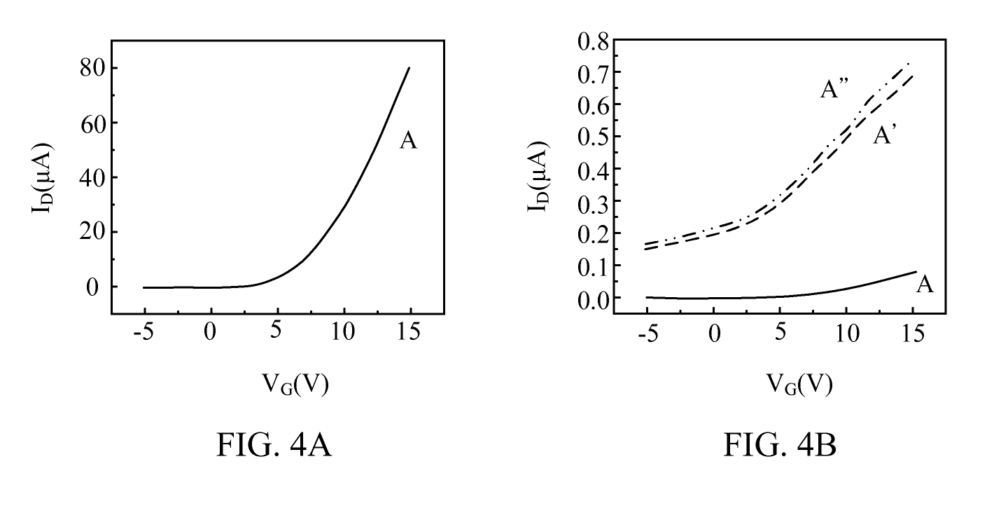

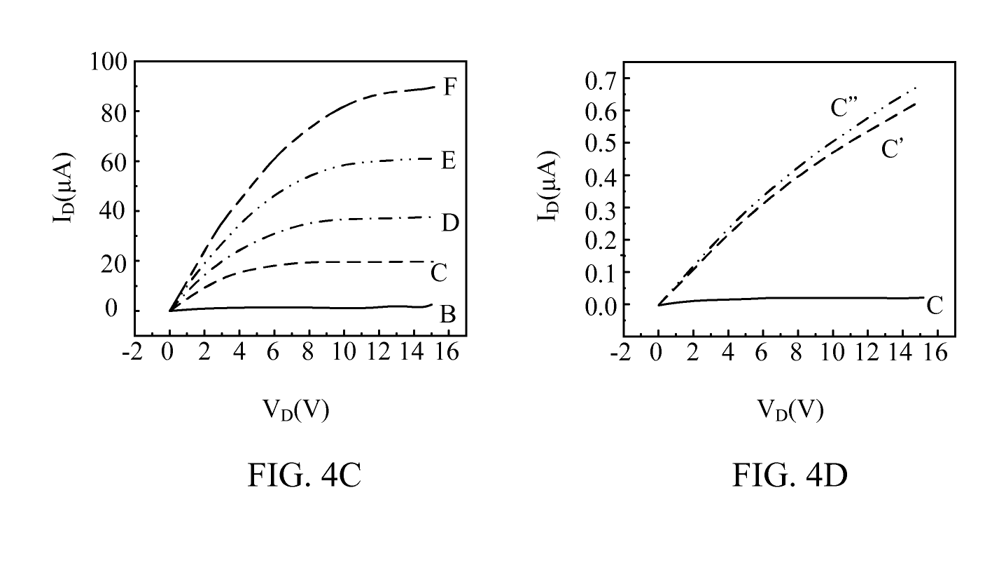

Please refer to FIGS. 2A, 2B and 2C again. Hereinafter, the detail processes of operating the biosensor 200 in the present invention will be described. First, the gate G and the drain D of the transistor 210 are applied voltage in advanced, so that the gate G and the drain D are relatively positive/negative electric potential to the source S. When the gate G-source S voltage (V_{GS}) is beyond the threshold voltage (Vₜₕ), a channel layer 211 is established between the insulating layer 212 and the passivation layer 213 and provides an electrical connection between the source S and the drain D, and a drain current is generated. Please refer to FIGS. 4A to 4D. FIG. 4A illustrates a drawing of drain currents versus gate voltages at this stage. The measured current is denoted as curve A. The nanostructures 222 are then attached to the sensing pad 221. The EGFR antibodies are applied to the sensing sink 223 for a determined time, for instance, 1 hour, to functionalize the sensor and then the sensing sink 223 is washed by a buffer solution, for instance, a phosphoric acid buffer solution, so that only the EGFR antibodies which are electrostatically attach on the nanostructures 222 are remained. The drain current is measured as a first current value at this time. Finally, a protein solution which includes the EGFR antigens (target protein) is applied to the sensing sink 223, so that the EGFR antigens are combined with the EGFR antibodies, therefore, the charge distribution of the cannel layer 211 is influenced by an electrical field caused by static electricity when the EGFR antigens combine with the EGFR antibodies, thus the drain current is measured as a second current value at this time. FIG. 4B illustrates a drawing of drain currents versus gate voltages for the biosensor 200 shown in FIG. 2C as structured in a functionalized biosensor having nanostructures. FIG. 4C illustrates a drawing of drain currents versus drain voltages for the prior bare biosensor described in FIG. 4A. FIG. 4D illustrates a drawing of drain currents versus drain voltages for the functionalized biosensor described in FIG. 4B. As shown in FIG. 4B, a curve A′ is demonstrated as a I_{D}-V_{G} curve for the functionalized biosensor as the biosensor 200 shown in FIG. 2C, and a curve A″ is demonstrated as another I_{D}-V_{G} curve for the functionalized biosensor with in which EGFR antibodies are added thereon. As shown in FIG. 4C and FIG. 4D, those curves B, C, D, E, and F are demonstrated as reference curves I_{D}-V_{D} for the prior bare biosensor in which the respective gate voltage is fixed at 2V, a curve C′ is demonstrated as a I_{D}-V_{D} curve of the functionalized biosensor, and a curve C″ is demonstrated as a I_{D}-V_{D} curve for the functionalized biosensor with addition of the EGFR antibodies. Thus, when the EGFR antibodies are applied to the sensing pad having the nanostructures (as shown in FIG. 2C), the electrical properties of the drain current are changed because of the static electricity which is induced in the gate of the transistor (as shown in FIG. 2A). A variation between the measured first current value and the measured second current value of the drain current is calculated so as to obtain the concentration of the target protein (that is, the EGFR antigens concentration) in the protein solution is revealed.

Because of the method of utilizing mutually corresponding proteins in the present invention, the biosensor in the present invention has high specificity. The biosensor may only detect a specific protein in a protein solution which may include various proteins.

In the present invention, by a dual gate structure of the transistor, the control of the gate voltage can be varied so that higher sensitivity can be obtained. When the magnitude of the measured first current value and the measured second current value of the drain current is very close, the user can adjust the gate voltage to a level to make the two current values have an accurate difference therebetween, so that the biosensor in the present invention has a great sensitivity. Furthermore, the biosensor does not only need less protein solution under test but also has a function of quick detection and a customized size of sensing pad or sensing sink, by way of the designated spacing between of the sensing area and the channel layer, and thereby prevents the influences from electromagnetic interference. In addition, the biosensor can be manufactured in volume base to reduce the cost for the users.

To sum up, the present invention has been disclosed as the preferred embodiments above, however, the above preferred embodiments are not described for limiting the present invention, various modifications, alterations and improvements can be made by persons skilled in this art without departing from the spirits and principles of the present invention, and therefore the protection scope of claims of the present invention is based on the range defined by the claims.

What is claimed is:

1. A biosensor with a dual gate structure for detecting a concentration of a target protein in a protein solution, the biosensor comprising:
a transistor having a gate, a source and a drain, wherein a channel layer is formed to establish an electrical connection between the source and the drain;
a sensing pad having a conductive area working as another gate and neighboring to the channel layer of the transistor, and a sensing area extended outward from the conductive area to be away from the channel layer of the transistor, wherein the gate and the conductive area of the sensing pad are separated from each other by the channel layer; and
wherein the sensing area is utilized to apply a first protein to generate a drain current value via the transistor, when the protein solution is applied on the sensing area to combine the first protein with the target protein and another drain current value is generated via the transistor, the concentration of the target protein in the protein solution is obtained from the difference between the two drain current values.

2. The biosensor of claim 1, wherein the sensing area and the transistor are surrounded by electrically isolating materials to separate the sensing area from the transistor and form a sensing sink in the sensing area to carry the proteins therein.

3. The biosensor of claim 1, wherein an insulating layer is disposed between the gate G and the channel layer.

4. The biosensor of claim 1, wherein a passivation layer is disposed between the conductive area and the channel layer.

5. The biosensor of claim 1, wherein the material of the sensing pad is the metal with good conductivity.

6. The biosensor of claim 1, wherein a plurality of nanostructures is attached to the sensing area for increasing combination ability to combine the first protein on the sensing area.

7. The biosensor of claim 6, wherein the nanostructures are ZnO nanorods, TiO₂ nanorods, and other types of materials which do not harm the proteins, by combination or alone.

8. A method of detecting a concentration of a target protein in a protein solution, the method comprising steps of:
forming a transistor having a gate, a source and a drain, wherein a channel layer is formed to establish an electrical connection between the source and the drain;
forming a sensing pad having a conductive area working as another gate and neighboring to the channel layer of the transistor, and a sensing area extended outward from the conductive pad to be away from the channel layer of the transistor, wherein the gate and the conductive area of the sensing pad are separated from each other;
attaching nano structures on the sensing area;
applying a specific voltage on the gate and the drain of the transistor, and the gate and the drain being relatively positive/negative electric potential to the source of the transistor;
applying first proteins on the sensing area, and measuring a first current value of the drain current;
applying the protein solution having the target protein on the sensing area, and measuring a second current value of the drain current; and
by a variation between the first current value and the second current value, obtaining the concentration of the target protein in the protein solution.

9. The method of claim 8, wherein the gate voltage is adjustable, when the magnitude of the first current value and the second current value is very close, the first current value is measured by adjusting the gate voltage to diversify the magnitude of the first current value and the second current value.

10. The method of claim 8, wherein the sensing area is sized on various user demands, and the gate and the conductive area of the sensing pad are separated from each other by the channel layer.

11. The method of claim 8, wherein the sensing area has nanostructures which are ZnO nanorod, TiO₂ nanorod, and other types of materials which do not harm the proteins, by combination or alone.

12. The method of claim 8, wherein the sensing area and the transistor are surrounded by electrically isolating materials to separate the sensing area from the transistor and form the sensing sink in the sensing area to carry the proteins therein.

13. A biosensor with a dual gate structure for detecting a concentration of a target protein in a protein solution, the biosensor comprising:
a transistor having a gate, a source and a drain, wherein a channel layer is formed to establish an electrical connection between the source and the drain;
a sensing pad having a conductive area working as another gate and neighboring to the channel layer of the transistor, and a sensing area extended outward from the conductive area to be far away from the channel layer of the transistor, wherein the gate and the conductive area of the sensing pad are separated from each other; and
wherein the sensing area is utilized to apply a first protein to generate a drain current value via the transistor, when the protein solution is applied on the sensing area to combine the first protein with the target protein and another drain current value is generated via the transistor, the concentration of the target protein in the protein solution is obtained by a variation between the two drain current values.
